(19) United States
(12) Patent Application Publication
(10) Pub. No.: US 2022/0039822 A1
(43) Pub. Date: Feb. 10, 2022

(54) DILATING INCISION DEVICE

(71) Applicant: Peking University Third Hospital (Third Clinical Medical College of Peking University), Beijing (CN)

(72) Inventors: Yonghui HUANG, Beijing (CN); Xiu'e YAN, Beijing (CN); Hong CHANG, Beijing (CN); Yaopeng ZHANG, Beijing (CN); Yingchun WANG, Beijing (CN); Wei ZHENG, Beijing (CN); Wenzheng LIU, Beijing (CN)

(21) Appl. No.: 17/127,322

(22) Filed: Dec. 18, 2020

(30) Foreign Application Priority Data
Aug. 6, 2020 (CN) 202010781206.8

(51) Int. Cl. A61B 17/3211

(52) U.S. Cl. CPC A61B 17/3211; A61B 2017/32113

(57) ABSTRACT

Disclosed is a dilating incision device, which relates to a surgical tool and includes an outer shell, a dilating element and an incision assembly. The dilating element that is arranged at one end of the outer shell and communicated with the inside of the outer shell is used to drill into and expand a stenosis segment of the pancreatic or biliary duct or a puncture path between lumens of the digestive tract. A sliding groove is formed in a side wall of the outer shell, by which the inner cavity of the outer shell is communicated with the outside. The incision assembly includes an incision knife arranged in the outer shell and a sliding handle housing the outer shell. An intercommunicating hole is formed in one end of the dilating element, by which the inner cavity of the dilating element is communicated. with the outside.

### TECHNICAL FIELD

The disclosure relates to a surgical tool, and in particular, to a dilating incision device.

### BACKGROUND

Endoscopic retrograde cholangiopancreatography (ERCP) and endoscopic ultrasonography (EUS) are two of the fastest developing technologies with the emerging of maximum new techniques in recent years. At present, most of benign diseases and part of malignant diseases of the biliary-pancreatic system can be diagnosed and treated by the two technologies and derived techniques thereof, which benefit many patients.

ERCP mainly involves inserting different accessories directed to different diseases through a therapeutic biopsy channel of a duodenoscope so as to achieve the purposes of diagnosis and treatment. However, some diseases cannot be treated by using the existing accessories, for example, some stenostic diseases of the pancreatic and biliary ducts. The stenosis in the pancreatic or biliary duct, whether benign or malignant, can be diagnosed and treated through the steps: (1) a duodenoscope reached ampulla, followed by cannulating a nampulla incision knife and a guidewire through an accessory channel, and then selective cannulation into the biliary duct and/or the pancreatic duct is carried out under the guidance of the guidewire; (2) a contrast agent is injected into the pancreatic and biliary ducts after the guidewire successfully enters the biliary duct and pancreatic duct system to identify the location and extent of the stenosis clearly; (3) if malignant stenosis is suspected in the biliary duct, biopsy is required in the stenosis segment to obtain pathological evidence; due to the special physiological structure, the pancreatic duct is likely to be injured after biopsy, resulting in severe complications such as acute pancreatitis, and malignant stenosis in the pancreatic duct can usually be diagnosed by preoperative imaging; and therefore, the pancreatic duct is generally not subjected to biopsy; and (4) in case of particularly severe stenosis with a diameter of less than 4.4 Fr (i.e., the ampulla incision knife cannot pass through the stenosis segment under the guidance of the guidewire), or even if the ampulla incision knife can narrowly pass through the stenosis segment, but cannot expand it to meet the requirement of next placement of a stent, the stenosis segment needs to be further subjected to dilating treatment. The existing dilation approach involves a step-by-step dilator (including Sohendra step-by-step dilation bougie and Sohendra stent retriever) and a step-by-step dilating balloon dilation. However, in clinical practice, some patients with severe stenosis, especially benign stenosis, can only choose surgeries and may receive interventional treatment under the guidance of EUS in some qualified hospitals because the above two dilation accessories cannot smoothly pass through and dilate the stenosis segments.

EUS is mainly used in three clinical aspects at present: (1) disease diagnosis by ultrasonic imaging; (2) fine needle aspiration biopsy or aspiration of tissues by insertion of a puncture needle through a biopsy channel of EUS and pathological disease diagnosis after obtaining the tissues; and (3) a series of interventional treatments under the guidance of EUS (mainly including: pseudocyst drainage under the guidance of EUS, biliary duct drainage under the guidance of EUS, pancreatic duct drainage under the guidance of EUS, gallbladder puncture drainage the guidance of EUS and gastroenterostomy under the guidance of EUS). In an interventional treatment under the guidance of EUS, it is necessary to perform the following steps: (1) a suitable puncture point is selected by EUS; (2) transgastric or transduodenal puncture into the target organ or lesion (the biliary duct, pancreatic duct, gall bladder, pseudocyst or jejunum) is performed by using a puncture needle under the guidance of EUS; (3) after a guidewire is detained in the target organ or lesion, Sohendra dilator is used to dilate the puncture channel under the guidance of the guidewire, or an incision knife is used solely to electrify and incise the puncture path, so that a drainage tube or stent is inserted through the puncture channel; and (4) after the puncture channel is dilated, the drainage tube and the stent are inserted under the guidance of X-rays and endoscopic ultrasonography. The implementation of the above step (3) is important to the success of subsequent treatment, but some patients have obvious fibrous scars along the puncture path, which prevent the dilator from smoothly entering the puncture channel, and since there are abundant small blood vessels along the puncture path, irreparable bleeding may be caused by sole use of the incision device and the purpose of safe dilating cannot be achieved.

### SUMMARY

The disclosure aims to provide a dilating incision device to solve the above problems in the prior art. The dilating incision device of the disclosure can pass through and dilate a severe stenosis segment of the pancreatic or biliary duct smoothly and can dilate a puncture path between lumens of the digestive tract.

To achieve the above objectives, the disclosure provides the following solutions: The disclosure provides a dilating incision device including an outer shell, a dilating element and an incision assembly. The dilating element that is arranged at one end of the outer shell and communicated with the inside of the outer shell is used to drill into and dilate a stenosis segment of the pancreatic or biliary duct and a puncture path between lumens of the digestive tract. A sliding groove is formed in a side wall of the outer shell, by which the inner cavity of the outer shell is communicated with the outside. The incision assembly includes an incision knife arranged in the outer shell and a sliding handle housing the outer shell. An intercommunicating hole is formed in one end of the dilating element, by which the inner cavity of the dilating element is communicated with the outside. The sliding handle is capable of driving the incision knife to move back and forth in an extension direction of the outer shell and causing the incision knife to extend out of the outer shell through the intercommunicating hole.

Preferably, the outer shell is in the form of a circular tube. The dilating element is cone-shaped and has a large end fixedly connected to the outer shell, with screw thread being formed on the outer periphery of the dilating element.

Preferably, the dilating element has a length of 2-3 cm.

Preferably, an electrical plug is arranged on the sliding handle and has one end electrically connected to an external power supply device and the other end fixedly connected to the incision knife to electrify the incision knife. The incision knife is needle-shaped.

Preferably, the sliding handle includes two circular rings and a connecting section housing the outer shell. The two circular rings are fixed to two ends of the connecting section, respectively, and arranged in a direction perpendicular to a length direction of the incision knife.

Preferably, a hand grid which is ring-shaped is arranged at an end, far away from the dilating element, of the outer shell to be held by an operator.

Preferably, scale marks are formed on the outer periphery of the outer shell, allowing an operator to observe a length by which the incision knife extends out of the intercommunicating hole.

Preferably, a limiting element is fixedly arranged at an end, close to the dilating element, of the outer shell, and houses the outer shell to limit the sliding handle, preventing disengagement or shifting of the sliding handle from the dilating element.

Compared with the prior art, the disclosure has achieved the following technical effects: The dilating incision device provided in the disclosure includes an outer shell, a dilating element, and an incision assembly. The dilating element that is arranged at one end of the outer shell and communicated with the inside of the outer shell is used to drill into and expand a stenosis segment of the pancreatic or biliary duct or a puncture path between lumens of the digestive tract, facilitating subsequent implantation of a surgical device. A sliding groove is formed in a side wall of the outer shell, by which the inner cavity of the outer shell is communicated with the outside.

The incision assembly includes an incision knife and a sliding handle. The incision knife is arranged in the outer shell, so that the incision knife can be retracted into the sliding groove when incision is not needed. The sliding handle sleeves the outer shell, facilitating direct application of a force to the sliding handle. An intercommunicating hole is formed in one end of the dilating element, by which the inner cavity of the dilating element is communicated with the outside and which allows the incision knife to extend in or out. The sliding handle can drive the incision knife to move back and forth in the extension direction of the outer shell and cause the incision knife to extend out of the outer shell through the intercommunicating hole. The ordinary stenosis can be mechanically expanded directly by the dilating element, which is relatively safe. In case of severe stenosis where the dilating element cannot directly pass through and expand the stenosis segment, the sliding handle is moved towards the dilating element, causing the incision knife to move towards the intercommunicating hole until the tip of the incision knife extends out of the intercommunicating hole. Subsequently, the incision knife is controlled to minimally incise the stenosis segment, and then retracted by moving the sliding handle after the completion of incising; meanwhile, the stenosis segment is expanded by the dilating element. Thus, the dilating of a severe stenosis segment or a puncture path can be achieved. Moreover, the dilating element allows a guidewire to pass therethrough, and then dilating and incision are performed under the guidance of the guidewire, which are safe and reliable.

### BRIEF DESCRIPTION OF THE DRAWINGS

To explain the technical solutions in examples of the disclosure or in the prior art more clearly, the accompanying drawings required in the examples will be briefly introduced below. Obviously, the accompanying drawings described below are merely some examples of the disclosure, and other accompanying drawings can be derived by a person of ordinary skill in the art from these accompanying drawings without creative work.

FIG. 1 is a front view of a dilating incision device in a dilating state according to an example of the disclosure.

FIG. 2 is a bottom view of the dilating incision device in FIG. 1.

FIG. 3 is a front view of a dilating incision device in an incising state according to an example of the disclosure.

FIG. 4 is a bottom view of the dilating incision device in FIG. 3.

In the figures: 1—outer shell, 11—sliding groove, 2—dilating element, 31—circular ring, 32—connecting section, 4—electrical plug, 5—incision knife, 6—limiting element, and 7—hand grip.

### DETAILED DESCRIPTION

The technical solutions in the examples of the disclosure will be described below clearly and completely in conjunction with the accompanying drawings in the examples of the disclosure. Obviously, the described examples are merely part of rather than all the examples of the disclosure. All other examples derived by a person of ordinary skill in the art from the examples in the disclosure without creative efforts shall fall within the protection scope of the disclosure.

An objective of the disclosure is to provide a dilating incision device to solve the technical problems of existing dilating accessories, such as failure to pass through a severe stenosis segment of the pancreatic or biliary duct and even failure to dilate the severe stenosis segment and a punch path between lumens of the digestive tract.

To make the above objective, features and advantages of the disclosure clearer and more comprehensible, the disclosure will be further described in detail below with reference to the accompanying drawings and specific examples.

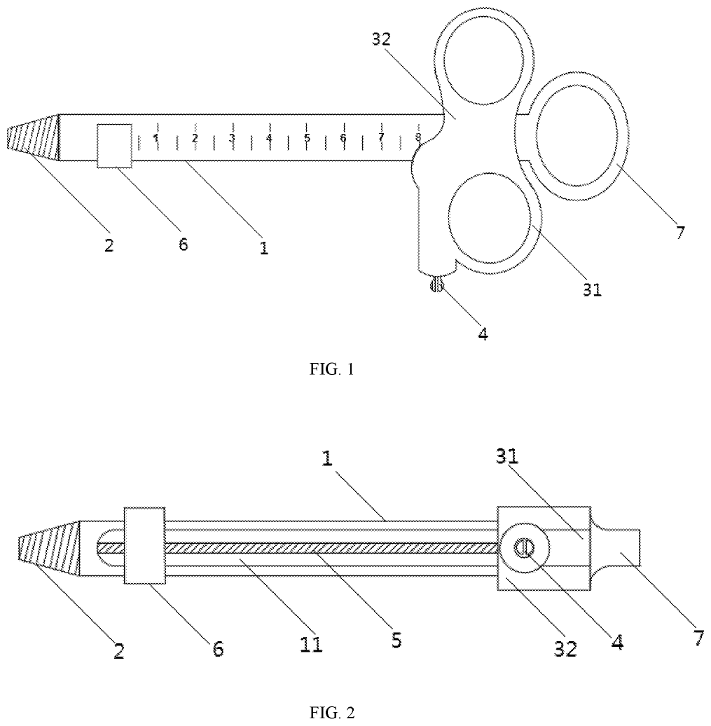

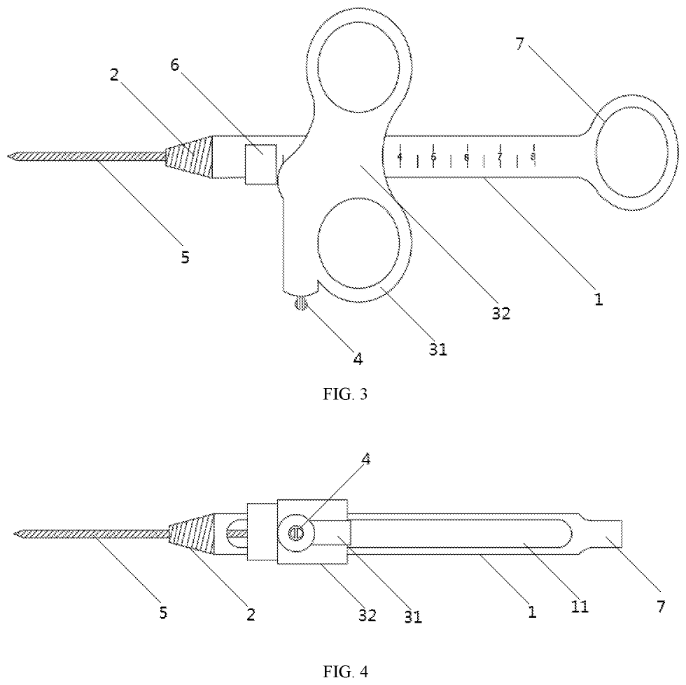

As shown in FIG. 1 to FIG. 4, the disclosure provides a dilating incision device including an outer shell 1, a dilating element 2, and an incision assembly. The dilating element 2 is arranged at one end of the outer shell 1 and communicated with the inside of the outer shell 1. The dilating element 2 is used to drill into and expand a stenosis segment of the pancreatic or biliary duct or a puncture path between lumens of the digestive tract, facilitating subsequent implantation of a surgical device. A sliding groove 11 is formed in a side wall of the outer shell 1, by which the inner cavity of the outer shell 1 is communicated with the outside. The incision assembly includes an incision knife 5 and a sliding handle. The incision knife 5 is arranged in the outer shell 1, so that when the incision knife 5 can be retracted into the sliding groove 11 when incision is not needed. The sliding handle sleeves the outer shell 1, facilitating direct application of a force on the sliding handle. An intercommunicating hole is formed in one end of the dilating element 2, by which the inner cavity of the dilating element 2 is communicated with the outside and which allows the incision knife 5 to extend in or out. The sliding handle can drive the incision knife 5 to move back and forth in the extension direction of the outer shell 1 and cause the incision knife 5 to extend out of the outer shell 1 through the intercommunicating hole. The ordinary stenosis can be mechanically expanded directly by the dilating element 2, which is relatively safe. During the mechanical dilating, the dilating incision device is in a dilating state (FIG. 1 to FIG. 2). In case of severe stenosis where the dilating element cannot directly pass through and expand the stenosis segment, the sliding handle is moved towards the dilating element 2, causing the incision knife 5 to move towards the intercommunicating hole until the tip of the incision knife 5 extends out of the intercommunicating hole. In this case, the dilating incision device is in an incising state (FIG. 3 to FIG. 4). Subsequently, the incision knife 5 is controlled to minimally incise the stenosis segment, and then retracted by moving the sliding handle after the completion of incising; meanwhile, the stenosis segment is expanded by the dilating element 2. Thus, the dilating of a severe stenosis segment or a puncture path can be achieved. Moreover, the dilating element allows a guidewire to pass therethrough, and then dilating and incision are performed under the guidance of the guidewire, which are safe and reliable.

Specifically, the outer shell 1 is in the form of a round tube. The dilating element 2 is cone-shaped and has a large end fixedly connected to the outer shell 1, so that the tip of the dilating element 2 can pass through the stenosis segment (or puncture path) firstly to expand gradually, thereby protecting the patient. The outer periphery of the dilating element 2 is provided with screw thread. In the actual dilating process, the outer shell 1 is rotated to drive the dilating element 2 to drill and expand spirally, so that the dilating resistance is reduced.

The dilating element 2 has a length of 2-3 cm. The dilating element has a diameter of 7 Fr or 8.5 Fr in a portion connected to the outer shell, and is provided with a 2 mm wide X-ray proof coating mark on an outer periphery portion connected to the outer shell. In the dilating state, the dilating incision device has a length of 200 cm, which, however, is merely a specific example, and the size of the dilating incision device provided in the disclosure is not limited thereto during actual production. A person skilled in the art can adaptively change various dimension as needed in actual dilating.

An electrical plug 4 is arranged on the sliding handle. One end of the electrical plug 4 is electrically connected to an external power supply device, and the other end of the electrical plug 4 is fixedly connected to the incision knife 5 to electrify the incision knife 5. When the incision knife 5 is held in place, the electrical plug 4 can be connected to the external power supply device to establish a closed circuit. A high-frequency current is used for incising, which is safe and reliable. The incision knife 5 is needle-shaped, which can realize minimal incision, thereby reducing the injury of a patient.

The sliding handle includes two circular rings 31 and a connecting section 32. The connecting section 32 houses the outer shell 1 and can slide back and forth along the outer periphery of the outer shell 1. The two circular rings 31 are fixed to two ends of the connecting section 32, respectively. In addition, the two circular rings 31 are arranged in a direction perpendicular to a length direction of the incision knife 5, which is convenient for an operator to hold. In use, the circular rings 31 can be held conveniently to directly apply a force by fingers passing therethrough.

A hand grip 7 is arranged at one end, far away from the dilating element 2, of the outer shell 1. The hand grip 7 is ring-shaped to be held by an operator. When spiral drilling of the dilating element 2 is performed, the hand grip 7 is directly acted upon by the fingers to drive the outer shell 1 to rotate, thereby causing the dilating element 2 to rotate. Thus, convenient operation is achieved. Preferably, the hand grip is provided with a guidewire hole by which the inside of the hand grip, the inner cavity of the outer shell and the inside of the dilating element are communicated, allowing a guidewire to pass through the dilating incision device smoothly.

Scale marks are formed on the outer periphery of the outer shell 2, allowing an operator to observe a length by which the incision knife 5 extends out of the intercommunicating hole. When the sliding handle drives the incision knife 5 to move, the sliding handle can correspondingly point to a scale value after it moves into any position, thereby ensuring that any extension length of the incision knife 5 can be read out and accurately. Thus, the length by which the incision knife 5 extends out of the intercommunicating hole can be calculated based on the scale mark corresponding to the position of the sliding handle. The protrusion length of the needle-shaped knife can be controlled effectively, allowing for safe and reliable operation. Preferably, the scale marks on the outer shell can also directly correspond to the extension lengths of the incision knife 5, that is, the scale mark corresponding to the position of the sliding handle is the extension length of the incision knife 5, allowing for more visual and convenient reading.

A limiting element 6 is fixed to an end, close to the dilating element 2, of the outer shell 1. The limiting element 6 sleeves the outer shell 1 to limit the sliding handle, preventing disengagement or shifting of the sliding handle from the dilating element 2. Moreover, whether the protrusion is in place can be determined, avoiding an instable protrusion length of the needle-shaped knife under manual control.

Specific examples are used herein to describe the principle and implementations of the disclosure. The foregoing description of the examples is only used to help understand the method of the disclosure and its core ideas; moreover, various modifications can be made by a person of ordinary skill in the art to specific implementations and application scope according to the ideas of the disclosure. To sum up, the contents of the specification shall not be construed as limitations to the disclosure.

What is claimed is:

1. A dilating incision device, comprising an outer shell, a dilating element and an incision assembly, wherein the dilating element that is arranged at one end of the outer shell and communicated with the inside of the outer shell is used to drill into and expand a stenosis segment of the pancreatic or biliary duct or a puncture path between lumens of the digestive tract; a sliding groove is formed in a side wall of the outer shell, by which the inner cavity of the outer shell is communicated with the outside; the incision assembly comprises an incision knife arranged in the outer shell and a sliding handle housing the outer shell; an intercommunicating hole is formed in one end of the dilating element, by which the inner cavity of the dilating element is communicated with the outside; the sliding handle is capable of driving the incision knife to move back and forth in an extension direction of the outer shell and causing the incision knife to extend out of the outer shell through the intercommunicating hole.

2. The dilating incision device according to claim 1, wherein the outer shell is in the form of a circular tube; and the dilating element is cone-shaped and has a large end fixedly connected to the outer shell, with screw thread being formed on the outer periphery of the dilating element.

3. The dilating incision device according to claim 2, wherein the dilating element has a length of 2-3 cm.

4. The dilating incision device according to claim 1, wherein an electrical plug is arranged on the sliding handle and has one end electrically connected to an external power supply device and the other end fixedly connected to the incision knife to electrify the incision knife; and the incision knife is needle-shaped.

5. The dilating incision device according to claim 1, wherein the sliding handle comprises two circular rings and a connecting section housing the outer shell; and the two circular rings are fixed to two ends of the connecting section, respectively, and arranged in a direction perpendicular to a length direction of the incision knife.

6. The dilating incision device according to claim 1, wherein a hand grip which is ring-shaped is arranged at an end, far away from the dilating element, of the outer shell to be held by an operator.

7. The dilating incision device according to claim 1, wherein scale marks are formed on the outer periphery of the outer shell, allowing an operator to observe a length by which the incision knife extends out of the intercommunicating hole.

8. The dilating incision device according to claim 1, wherein a limiting element is fixedly arranged at an end, close to the dilating element, of the outer shell, and houses the outer shell to limit the sliding handle, preventing disengagement or shifting of the sliding handle from the dilating element.
